(19) United States
(12) Patent Application Publication
(10) Pub. No.: US 2024/0156698 A1
(43) Pub. Date: May 16, 2024

(54) BIOCIDE COMPOSITIONS AND USES THEREOF

(71) Applicant: PHARMOTECH SA, EYSINS (CH)

(72) Inventor: RODIN AESCHBACH, MEYRIN (CH)

(21) Appl. No.: 18/278,428

(22) PCT Filed: Feb. 23, 2022
(86) PCT No.: PCT/EP2022/054474

§ 371 Date: Aug. 23, 2023

(30) Foreign Application Priority Data
Feb. 23, 2021 (EP) 21158789.4

(51) Int. Cl. A61K 8/34; A61Q 17/00

(52) U.S. Cl. CPC A61K 8/34; A61Q 17/005; A61K 2800/49; A61K 2800/70

(57) ABSTRACT

The present invention is directed to long lasting topical liquid hydroalcoholic biocide compositions characterised by contents of cannabidiol (CBD) and a CBD solubilizing agent, preferably propylene glycol.

### FIELD OF THE INVENTION

The present invention relates to the field of biocides. In particular, the invention relates to biocide formulations useful in hand-sanitization.

### BACKGROUND OF THE INVENTION

Hand hygiene is of utmost importance as it may be contaminated easily from direct contact with airborne microorganism droplets. Particularly in situations like pandemic outbreak, it is crucial to interrupt the transmission chain of the virus by the practice of proper hand sanitization. Maintaining good hand hygiene in hospital settings and in public has become of importance more than ever. Effective hand disinfecting agents formulated in various types and forms such as antimicrobial soaps, water-based or alcohol-based hand sanitizer, with the latter being widely used in hospital settings have been placed in the front scene of development efforts recently. To date, most of the effective hand sanitizer products are alcohol-based formulations containing 62%-95% of alcohol as it can denature the proteins of microbes and the ability to inactivate viruses. With the emergence of SARS-CoV-2, it became even more crucial to interrupt the transmission chain of the virus through strict infection control tools and following face masks, appropriate hand hygiene is of utmost importance as hands may be contaminated from direct contact with patients' Jane Lee Jia Jing et al., 2020, Int. J. Environ. Res. Public Health 2020, 17, 3326. However, the current hydroalcoholic formulations only have a limited time effect on the skin and applications need to be reiterated very often to maintain a proper hand sanitization. Therefore, the is a need for developing new hand sanitization formulation with a longer lasting effect.

### SUMMARY OF THE INVENTION

The present invention relates to the unexpected finding that cannabidiol (CBD) is able to prolong the biocide effect of hydroalcoholic solutions. In particular, CBD was able to expand the biocidal effect up to at least 1 or 2 hours compared to one minute.

An aspect of the invention provides a topical liquid hydroalcoholic composition comprising CBD for use as a long-acting biocide, wherein said liquid hydroalcoholic composition comprises 0.01 to 10% w/w CBD, a lower alcohol having from 2 to 4 carbon atoms from about 60 to about 90% w/w, a CBD solubilizing agent from 2 to 25% w/w and water in a quantity sufficient for the composition to total 100% w/w, wherein the quantity of water is not lower than 1% and wherein the said CBD solubilizing agent is miscible with the lower alcohol.

An aspect of the invention provides a skin sanitizing composition comprising a topical liquid hydroalcoholic composition according to the invention.

An aspect of the invention provides a topical liquid hydroalcoholic composition comprising 0.01 to 10% w/w CBD, a lower alcohol having from 2 to 4 carbon atoms from about 60 to about 90% w/w, a CBD solubilizing agent from 2 to 25% w/w and water in a quantity sufficient for the composition to total 100% w/w, wherein the quantity of water is not lower than 1% and wherein the said CBD solubilizing agent is miscible with the lower alcohol.

Another aspect of the invention relates to a method for the preparation of a biocide composition, said method comprising the steps of
Providing CBD solubilized in lower alcohol having from 2 to 4 carbon atoms;
Mixing said solubilized CBD with a CBD solubilizing agent wherein the said CBD solubilizing agent is miscible with the lower alcohol having from 2 to 4 carbon atoms;
Adding water, wherein said biocide composition consists in a liquid hydroalcoholic composition comprising 0.01 to 10% w/w CBD, a lower alcohol having from 2 to 4 carbon atoms from about 60 to about 90% w/w, a CBD solubilizing agent from 2 to 25% w/w and water in a quantity sufficient for the composition to total 100% w/w, wherein the quantity of water is not lower than 1% and wherein the said CBD solubilizing agent is miscible with the lower alcohol.

Another aspect of the invention relates to a method for disinfecting the skin of a subject, said method comprising applying a biocide composition of the invention to a skin surface of said subject, wherein the composition, after application to said skin tissue, provides disinfectant properties for at least 2 hours after application of the composition to the skin tissue.

### DETAILED DESCRIPTION OF THE INVENTION

Examples of “a lower alcohol having from 2 to 4 carbon atoms” comprise ethanol, isopropanol, and butanol.

The term “CBD solubilizing agent” refers to an agent that is able to solubilize CBD. Suitable solubilizing agents are agents that are miscible with a lower alcohol having from 2 to 4 carbon atoms, in particular ethanol. Example of solubilizing agents can be a propane diol (propane-1,2 diol) such as propylene glycol or a propane triol (propane 1,2,3-triol) such as glycerin and the like.

The term “biocide” characterizes an agent that exhibit a biocidal activity. Biocidal activity can be measured as described herein.

The term “liquid” characterizes a composition that is in liquid state at room temperature under atmospheric pressure. Typically, the dynamic viscosity of a liquid composition according to the invention is between 0.8 and 3.05 cP at 20° C.

The term “cannabidiol (CBD)” refers to a type of cannabinoid that can be found in cannabis plant having the following chemical structure:

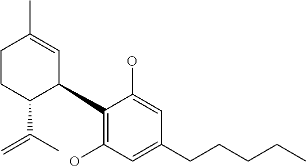 [SMILES: C=C(C)[C@@H]1CCC(C)=C[C@H]1c1c(O)cc(CCCCC)cc1O]

2-[(1R,6R)-6-isopropenyl-3-methylcyclohex-2-en-1-yl]-5-pentylbenzene-1,3-diol, also named s Δ²-cannabidiol.

It is a major constituent of the Cannabis plant, second to THC, and represents up to 40% in its extracts. Compared with THC has a very low affinity for CB1 and CB2 receptors which results in this substance being non-psychoactive. CBD can be extracted from various Cannabis plant species including Cannabis sativa, indica and ruderalis. In particular, CBD can be extracted as a pure compound from genetically modified cannabis plant which is producing increased levels of CBD as compared to naturally occurring plants.

According to one embodiment, is provided a CBD of a natural origin, that is extracted from Cannabis strains variety.

According to another embodiment, a CBD can be isolated by standard methods known to the skilled person, for example comprising collecting of plant material and extraction and purification.

Alternatively, CBD may be prepared by synthetic methods.

### Compositions According to the Invention

According to a particular aspect is provided a topical hydroalcoholic liquid composition comprising 0.01 to 9% w/w CBD, a lower alcohol having from 2 to 4 carbon atoms from about 60 to about 90% w/w, a CBD solubilizing agent from 2 to 25% w/w and water in a quantity sufficient for the composition to total 100% w/w, wherein the quantity of water is not lower than 1% and wherein the said CBD solubilizing agent is miscible with the lower alcohol. According to particular aspect, the composition of the invention is a sanitizing composition, in particular hand-sanitizing composition.

According to a particular aspect, compositions of the present invention are useful for inactivating viruses and microorganisms.

According to a particular aspect, compositions of the present invention have a long-acting effect on the inactivation of viruses and microorganisms. Typically, the biocide effect of compositions of the present invention lasts up to at least 1 or 2 hours.

According to a particular aspect, compositions of the present invention have a long-acting biocide effect when applied to a skin surface.

Compositions of this invention may further comprise one or more pharmaceutically acceptable additional ingredient(s) such as alum, stabilizers, antimicrobial agents, buffers, coloring agents, flavoring agents, adjuvants, and the like.

Compositions of the invention and unit dosages thereof, and in such form may be employed as liquids such as solutions, suspensions, emulsions, elixirs. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Liquid forms suitable for topical administration may include a suitable aqueous or non-aqueous vehicle with buffers, suspending and dispensing agents, colorants, flavors and the like. According to a particular aspect, a composition according to the invention contains from about 0.1 to 5% (weight (w)/weight (w)) CBD (e.g. from about 0.2% to 5% w/w for example 0.2% to 5% w/w or from about 0.2 to about 0.5% w/w).

According to another further particular aspect, the lower alcohol is ethanol.

According to another further particular aspect, the lower alcohol is isopropanol.

According to another further particular aspect, the lower alcohol is butanol.

According to another further particular aspect, a composition according to the invention contains from about 60 to 90% (w/w) ethanol, such as 70 to 90% (e.g. 70%% w/w).

According to another further particular aspect, the CBD solubilizing agent is propylene glycol.

According to another further particular aspect, a composition according to the invention contains from about 2 to 25% (w/w) propylene glycol, preferably from about 3 to 25% or 5 to 25%, for example 3 to 20% or 5 to 20% (e.g. 20% w/w).

According to another further particular aspect, a composition according to the invention contains from about 1 to 25% (w/w) water (e.g. 9.5 w/w).

According to another further particular aspect, a hydroalcoholic composition according to the invention contains CBD 0.5% (w/w), ethanol 70%, propylene glycol 20% and water 9.5%.

### Methods and Uses According to the Invention

According to a particular embodiment, are provided a composition thereof for use as biocide or sanitizing compositions.

Compositions of this invention may also be applied topically to the skin, in particular locally for example by a local spray of a formulation according to the invention.

Another aspect of the invention relates to a method for the preparation of a biocide composition, as described herein.

According to a particular embodiment, is provided a method for the preparation of a biocide composition wherein CBD solubilized in lower alcohol having from 2 to 4 carbon atoms is subjected to heating under steam atmosphere for about 30 min.

According to a particular embodiment, is provided a method for the preparation of a biocide composition wherein CBD solubilized in lower alcohol having from 2 to 4 carbon atoms is further added with a CBD solubilizing agent and mixed for about 30 minutes.

According to a particular embodiment, is provided a method for the preparation of a biocide composition wherein the mixture of solubilized CBD with a CBD solubilizing agent is further mixed after water addition for about 30 minutes.

According to a particular embodiment, CBD is solubilized in a lower alcohol having from 2 to 4 carbon atoms before adding to the CBD solubilizing agent (e.g. PG) at a concentration of about 2 g or 5 g or 10 g per 1 kg of final solution. It is important that CBD is not mixed with water first but that water is added to the final mixture. For example, for 1 kg of final solution, 5 g or 10 g of CBD are solubilized in 700 g of ethanol with 5 to 20 g of PG. Water is added at the final stage of preparation.

References cited herein are hereby incorporated by reference in their entirety. The present invention is not to be limited in scope by the specific embodiments and drawings described herein, which are intended as single illustrations of individual aspects of the invention, and functionally equivalent methods and components are within the scope of the invention.

### EXAMPLES

The following abbreviations refer respectively to the definitions below:
CBD (cannabidiol).

### Example 1: Preparation of a Composition of the Invention

A composition of the invention was prepared as described below:

0.5 g of CBD is dissolved in 70 g ethanol and then 20 g of polypropylene glycol (PG) is added to the mixture which is then mixed from about 30 min to 1 h 30 min at 20 C.° to obtain a clear solution and finally 100 g of water QSad was added (Formulation A).

Further formulations 1 to 7 were prepared as follows in Table 1 below:

TABLE 1

| | 1 | 2 | 3 | 4 | 5 | 6 | 7 |
|---|---|---|---|---|---|---|---|
| CBD | 0.5 | 0.5 | 0.5 | 0.5 | 0.5 | 0.5 | 0.5 |
| PG | 2 | 5 | 10 | 20 | 25 | 5 | 10 |
| Ethanol | 60 | 60 | 60 | 60 | 60 | 70 | 70 |
| Water | 37.5 | 34.5 | 29.5 | 19.5 | 14.5 | 24.5 | 19.5 |

Amounts are expressed in % w/w

### Example 2: Use of a Composition of the Invention as a Biocide on a Steel Surface

The composition (100 μL) prepared according to Example 1 was applied onto a steel surface. After that, a bacterial test suspension (1.0×10⁸ and 5.0×10⁸ ufc/ml, determined by spectrophotometry) is applied to the steel surface. The surface is kept at the specified temperature for a defined period of time as detailed below. Various contact times between the microorganism and the surface coated with the composition of the invention are tested: 1 minute, 1 hour and 2 hours at room temperature.

The culture media which are used are dehydrated media purchased from certified suppliers and the media is prepared in strict accordance with the manufacturer's instructions and according to the corresponding standard. The preparation batches of these media are positively and negatively controlled after sterilization, as well as the diluents used.

The strains are obtained from the Spanish Type Culture Collection (CECT), kept frozen and sown in the culture media as indicated in the procedure AVT/09 (internal procedure on how to keep frozen the strains obtained from the Spanish Type Culture Collection (CECT), how to sown in the culture media which is aligned with the indications of the European Standard EN 12353 regarding the methods for the preservation of test microorganisms and determination of bactericidal, mycobactericidal, sporicidal and fungicidal activity of chemical disinfectants and antiseptics established by CEN/TC 216. Pseudomonas aeruginosa (CECT 116), Staphylococcus aureus (CECT 239), Escherichia coli (CEC T405) were used. Viable microorganisms were used from microbial stock kept at −80° C. To do this, fresh culture of each microorganism is generated from this stock by plate sowing. Pseudomonas aeruginosa or Staphylococcus aureus in Soy Triptone Agar at 37° C.±1° C. for 18 to 24 hours.

The surface is transferred to a previously validated neutralization medium so that the action of the disinfectant is immediately neutralized and incubated during 24 hours at 37° C. The number of surviving microorganisms that can be recovered from the surface is determined quantitatively. The determination of viable cells was carried out by serial dilutions up to 10-4 and plating method on specific media and afterwards incubated according to the conditions specified for the microorganism. Finally, the number of bacteria in relation to the colonies grown, the CFU (Colony Forming Units) on the agar surface steel was determined. The percentage of viability reduction is calculated for each microorganism, using the following formula: Reduction % (CFU/ml)=((B−A)/B)×100, where, A: mean of viable cells after contact time with tested product. B: mean of viable cells in CONTROL after contact time. The percentages of reduction of the microorganisms are listed in Table 2 below.

TABLE 2

| | | % REDUCTION | % REDUCTION | % REDUCTION |
|---|---|---|---|---|
| Contact | | Pseudomonas | Staphylococcus | Escherichia |
| time | | aeruginosa | aureus | Coli |
| 1 | min | 91.4545 | 86.9845 | 83.3000 |
| 1 | hour | 99.9942 | 99.9994 | 99.9986 |
| 2 | hours | 99.2000 | 99.9984 | 99.9486 |

The microbial inhibition system for the analyzed formulation of the invention presents bactericidal residual activity with a reduction percentage of 99.20% after 2 hours against Pseudomonas aeruginosa, 99.99% against Staphylococcus aureus and 99.94% against Escherichia coli which is an extremely surprising long-lasting activity for alcoholic solutions used as biocides or sanitizing solutions which generally are not efficient more than a minute.

The percentages of reduction of Pseudomonas aeruginosa are listed in Table 3 below for further formulations of the invention, depending on their content in PG.

TABLE 3

Contact time

| Formulations | 1 hour | 2 hours |
|---|---|---|
| 1 | 16.49 | 79.70 |
| 2 | 92.62 | 99.99 |
| 3 | 79.20 | 99.99 |
| 4 | 56.40 | 99.99 |
| 6 | 44.66 | 95.75 |
| 7 | 85.88 | 99.99 |

All the formulations with more than 2% PG reduced P. aeruginosa 2 h more than 99.2%. PG content shows to have a positive effect on the long-lasting effect towards the reduction of P. aeruginosa. Lower levels of PG (2%) and 60% ethanol present a less pronounced long-lasting effect compared to the other formulations of the invention.

The percentages of reduction of Staphylococcus aureus are listed in Table 4 below for further formulations of the invention, depending on their content in PG.

TABLE 4

Contact time

| Formulations | 1 hour | 2 hours |
|---|---|---|
| 1 | 75.97 | 99.81 |
| 2 | 65.67 | 99.90 |
| 3 | 48.98 | 99.99 |
| 4 | 98.81 | 99.99 |
| 5 | 99.52 | 99.99 |
| 6 | 97.15 | 99.99 |
| 7 | 99.31 | 99.55 |

All the formulations with at least 2% PG reduced Staphylococcus aureus after 2 h more than 99.5%.

The long-lasting activity on a steel surface is representative of the possible long-lasting activity on human skin and it is expected to be transposable to the activity on human skin for example after hand washing with a composition of the invention.

### Example 3: Skin Absorption after Application of a Composition According to the Invention

The skin absorption of a composition of the invention (as described in Example 2) was studied by determining the amount of CBD permeated, deposited in the stratum corneum and the rest of the skin (only the diffusion area) after application of a formulation of the invention to the skin: 2.38±1.52 μg CBD/cm² were absorbed in the skin, which is the 15.85% of the CBD applied on the top of the skin at the beginning of the experiment.

The transdermal absorption tests are carried out using a modified methodology of Franz diffusion cells, in which a semi-permeable membrane is used, such as reconstructed skin or skin explants. The membrane is located between the (i) donor and (ii) receptor compartments with the stratum corneum facing upward. The product is applied to the exposed stratum corneum in the donor compartment. Under the membrane, the receiving chamber contains a solution that simulates physiological conditions and where the tested substances are highly soluble.

After the product is delivered to the surface of the skin, samples are taken from the receptor compartment at predetermined times to determine the amount of substance accumulated in the receptor compartment as a function of time. In addition, at the end of the test, the extraction of the substance that has been retained in the skin is carried out. For the quantification of all the samples obtained, a suitable analytical method is necessary for each substance investigated. The skin explants were dermatomed (the dermatome device allows to cut skin layers of the skin at a determined thickness) at 200 μm thickness and placed on Franz cells, the receptor compartment was filled with PBS solution, used to simulate the physiological fluid and allowed to equilibrate for at least 30 minutes. Then, receptor solution was added into the donor compartment and the integrity of the skin was determined. After removing the solution and drying the donor, the composition of the invention 3 μl/0.38 cm2 (7.9 uL/cm2) was administered into the donor compartment.

Samples from the receptor compartment were taken at predetermined times to obtain the amount of active permeated. Said samples were filtered on 0.2 μm pore size cellulose acetate filters and analyzed by HPLC. The sample volume taken was replaced with fresh receptor solution. After taking the last sample from the receptor compartment, a first wash of the skin surface was performed with PBS, then, the Franz cells were disassembled.

The skin was carefully dried and the stratum corneum was removed by tape stripping. Tapes were put in 2 mL of extraction medium for at least 2 hours. The skin diffusion area was cut into small pieces and placed in extraction liquid under continuous stirring for 1 hour. After filtering the samples obtained, they were analyzed by HPLC to obtain the amount of substance retained on the skin.

No permeation of CBD was found 1 and 2 hours after the application of the composition of the invention to the skin. The amount of CBD contained on the stratum corneum was quantified at 2 hours after the stratum corneum was removed by tape stripping. No CBD was found in the stratum corneum after 2 hours after the application of the composition of the invention to the skin.

Therefore, the long-lasting effect is due to the remaining of CBD in surface of the skin it is applied to without permeation/absorption into the skin.

1-16. (canceled)

17. A topical liquid hydroalcoholic composition comprising CBD, wherein said topical liquid hydroalcoholic composition comprises 0.1% to 5% w/w CBD, a lower alcohol having from 2 to 4 carbon atoms from about 60% to about 90% w/w, a CBD solubilizing agent from 2% to 25% w/w and water in a quantity sufficient for the composition to total 100% w/w, wherein the quantity of water is not lower than 1% and wherein the said CBD solubilizing agent is miscible with the lower alcohol, wherein said composition is a long-acting biocide.

18. The topical liquid hydroalcoholic composition according to claim 17, wherein said composition contains from about about 0.2% to about 5% w/w CBD.

19. The topical liquid hydroalcoholic composition according to claim 17, wherein the lower alcohol is ethanol.

20. The topical liquid hydroalcoholic composition according to claim 17, wherein the lower alcohol is isopropanol.

21. The topical liquid hydroalcoholic composition according to claim 17, wherein said composition contains from about 60% to 90% (w/w) ethanol.

22. The topical liquid hydroalcoholic composition according to claim 17, wherein the CBD solubilizing agent is propylene glycol.

23. The topical liquid hydroalcoholic composition according to claim 17, wherein said composition contains from about 3% to 25% (w/w) propylene glycol.

24. The topical liquid hydroalcoholic composition according to claim 17, wherein said composition contains from about 1% to 25% (w/w) water.

25. The topical liquid hydroalcoholic composition according to claim 17, wherein said hydroalcoholic composition contains CBD 0.5% (w/w), ethanol 70% (w/w), propylene glycol 20% (w/w), and water 9.5% (w/w).

26. The topical liquid hydroalcoholic composition according to claim 17, wherein the dynamic viscosity of said composition is between 0.8 and 3.05 cP at 20° C.

27. A sanitizing topical liquid hydroalcoholic composition comprising 0.1% to 5% w/w CBD, a lower alcohol having from 2 to 4 carbon atoms from about 60% to about 90% w/w, a CBD solubilizing agent from 2% to 25% w/w and water in a quantity sufficient for the composition to total 100% w/w, wherein the quantity of water is not lower than 1% and wherein the said CBD solubilizing agent is miscible with the lower alcohol.

28. The topical liquid hydroalcoholic composition according to claim 27, wherein said composition is a skin sanitizing composition.

29. The topical liquid hydroalcoholic composition according to claim 27, wherein said composition contains from about 0.5% to about 1% w/w CBD.

30. The topical liquid hydroalcoholic composition according to claim 28, wherein the lower alcohol is ethanol.

31. The topical liquid hydroalcoholic composition according to claim 27, wherein said composition has a long-acting biocide effect when applied to a skin surface.

32. A method for the preparation of a biocide composition, said method comprising the steps of:
providing CBD solubilized in lower alcohol having from 2 to 4 carbon atoms;
mixing said solubilized CBD with a CBD solubilizing agent wherein the said CBD solubilizing agent is miscible with the lower alcohol having from 2 to 4 carbon atoms;
adding water, wherein said biocide composition comprises a liquid hydroalcoholic composition comprising 0.1% to 5% w/w CBD, a lower alcohol having from 2 to 4 carbon atoms from about 60% to about 90% w/w, a CBD solubilizing agent from 2% to 25% w/w and water in a quantity sufficient for the composition to total 100% w/w, wherein the quantity of water is not lower than 1% and the said CBD solubilizing agent is miscible with the lower alcohol having from 2 to 4 carbon atoms.
